(19) United States
(12) Patent Application Publication
(10) Pub. No.: US 2026/0071161 A1
(43) Pub. Date: Mar. 12, 2026

(54) MICROFLUIDIC CHIP TO MODEL MULTI-ORGAN INTERACTIONS

(71) Applicant: University of Virginia Patent Foundation, Charlottesville, VA (US)

(72) Inventors: Rebecca Rose Pompano, Charlottesville, VA (US); Sophie Reed Cook, Charlottesville, VA (US); Jennifer M. Munson, Salem, VA (US); Jennifer H. Kennedy, Roanoke, VA (US)

(21) Appl. No.: 19/267,203

(22) Filed: Jul. 11, 2025

Related U.S. Application Data
(60) Provisional application No. 63/693,496, filed on Sep. 11, 2024.

(51) Int. Cl. C12M 3/06; C12M 1/00; C12M 1/32

(52) U.S. Cl. CPC C12M 23/16; C12M 23/12; C12M 29/00

(57) ABSTRACT

The present disclosure provides for devices, systems, and methods of using microfluidic chips to model multi-organ interactions. The microfluidic chip includes a first pump configured to pump a first fluid flowing through the microfluidic chip via a first channel and a second pump configured to pump a second fluid flowing through the microfluidic chip via a second channel. The microfluidic chip further includes a barrier well, including a barrier cell culture insert disposed in the barrier well. Additionally, the microfluidic chip includes the first channel connecting the first pump to a first barrier inlet and connecting a first barrier outlet to the first pump and the second channel connecting the second pump to a second barrier inlet and connecting a second barrier outlet to the second pump.

### CROSS-REFERENCE TO RELATED APPLICATIONS

This application claims the benefit of U.S. Provisional Application entitled “MULTI-MEDIA, MULTI-ORGAN-ON-CHIP TO MODEL BRAIN-IMUNE INTERACTIONS IN NEURINFLAMMATION AND NEURODEGENERATION” and having Ser. No. 63/693,496, filed Sep. 11, 2024, which is herein incorporated by reference in its entirety.

### STATEMENT OF GOVERNMENT INTEREST

This invention was made with government support under AG071661, AI174207, AI131723, and EB029127 awarded by the Institutes of Health. The government has certain rights in this invention.

### BACKGROUND

Over the last few decades, researchers have developed ever more sophisticated tools to study how organs transmit cellular and molecular signals throughout a living organism. The most common way to study inter-organ communication is through the use of animal models, and these have been powerful tools to determine biological mechanisms and test drug efficacy and toxicology. However, animal models suffer from the challenge of isolating the communication between any particular subset of organs, challenges in analyzing functions of internal organs such as the brain over time, and inevitable species differences compared to humans. Biological barriers can be particularly important in studying communication between organs. One example is the communication of the central nervous system with organs in the periphery of the body (e.g. outside of the central nervous system). For example, in vivo antigens in the brain are picked up by interstitial fluid flow, enter the meningeal lymphatics or other drainage pathways, and drain to cervical lymph nodes. Meanwhile, cells from the lymph node enter the blood stream and may eventually cross the blood-brain-barrier to enter the brain, so there is reciprocal cellular and molecular communication through two types of barriers between the lymph node and the brain. In instances of neuroinflammation and neurodegeneration, brain-lymph node communication is still poorly understood and challenging to study using traditional mouse models.

### SUMMARY

In accordance with the purpose(s) of this disclosure, as embodied and broadly described herein, the disclosure, in various aspects, relates to a microfluidic device to model multi-organ interactions and methods of use thereof. Aspects of the present disclosure provide for a microfluidic device, comprising: a first pump; a first well comprising a first cell culture insert disposed in the first well; and a first channel connecting the first pump to the first well.

The present disclosure also provides for methods for modeling multi-organ interactions, comprising: flowing a first fluid through a microfluidic chip, the microfluidic chip, comprising: a first pump configured to pump the first fluid through the microfluidic chip; a first well, the first well comprising a first cell culture insert disposed in the first well; and a first channel connecting the first pump and the first well, wherein the first pump is in fluidic communication with the first well via the first fluid.

In some aspects, the present disclosure provides for a system, comprising: a microfluidic chip, comprising: a first pump configured to pump a first fluid flowing through the microfluidic chip via a first channel; a second pump configured to pump a second fluid flowing through the microfluidic chip via a second channel; a barrier well, comprising a barrier cell culture insert disposed in the barrier well; the first channel connecting the first pump to a first barrier inlet and connecting a first barrier outlet to the first pump; and the second channel connecting the second pump to a second barrier inlet and connecting a second barrier outlet to the second pump.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a schematic and FIG. 1B is an image of a single-media, microfluidic chip to model biological cultures in isolation according to various embodiments of the present disclosure.

FIG. 2A is a schematic and FIG. 2B is an image of a microfluidic chip with a barrier to model biological barriers with two medias according to various embodiments of the present disclosure.

FIG. 3A is a schematic and FIG. 3B is an image of a multi-media chip to model biological interactions across barriers according to various embodiments of the present disclosure.

FIG. 4 is a schematic of an example of a microfluidic, multi-media chip to model multi-organ interactions according to various embodiments of the present disclosure.

FIG. 5A is a cross-sectional view of a well of a microfluidic chip according to various embodiments of the present disclosure.

FIG. 5B is a cross-sectional view of a barrier well of a microfluidic chip according to various embodiments of the present disclosure.

FIG. 6 illustrates an exploded view of various components of a well of a microfluidic chip according to various embodiments of the present disclosure.

FIG. 7A depicts the geometry of a well of a microfluidic chip with a layer of cells present on the cell culture insert according to various embodiments of the present disclosure.

FIG. 7B depicts a table of the goal, predicted average, and predicted maximum velocity through the layer of cells and the corresponding channel velocities for various tissue models according to various embodiments of the present disclosure.

FIG. 7C illustrates the predicted velocity in the layer of cells at a range of channel speeds for a brain well model according to various embodiments of the present disclosure.

FIG. 7D illustrates the predicted velocity in a layer of cells at cutlines in the top, middle, and bottom of the layer of cells for a naïve brain model well, an Alzheimer's disease brain model well, and a lymph node paracortex model well according to various embodiments of the present disclosure.

FIG. 8A depicts the geometry of a barrier well of a microfluidic chip with a layer of cells on the top and the bottom of the cell culture insert according to various embodiments of the present disclosure.

FIG. 8B is a schematic illustration of a flow regime across a barrier without and with a plug according to various embodiments of the resent disclosure.

FIG. 8C depicts a table of different channel velocities and the corresponding goal, predicted average, and predicted maximum velocity across a barrier for the first loop and the second loop according to various embodiments of the present disclosure.

FIG. 8D illustrates the predicted velocity along the barrier across a y and x cutline in a top chamber of a barrier well without a plug and with a plug and in a bottom chamber of a barrier well according to various embodiments of the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, microfluidic and biological modeling techniques and the like, which are within the skill of the art. Such techniques are explained fully in the literature.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### DISCUSSION

The present disclosure provides for microfluidic devices and chips, systems including microfluidic chips, and methods of using microfluidic chips to model multi-organ interactions, and the like.

Various embodiments of the present disclosure provide for a multi-media, multi-organ-on-chip to model multi-organ interactions such as brain-immune interactions in neuroinflammation and neurodegeneration. According to various embodiments of the present disclosure, microfluidic chips can provide an experimentally accessible platform to study communication by connection multiple tissue models using biomimetic fluid flow. In some examples, the microfluidic chip of the present disclosure can be used to co-culture tissue engineered models of the brain, meningeal lymphatics or blood-brain-barrier, and lymph node paracortex under two distinct media loops. For example, a system including the microfluidic chip can be used to model communication between the brain and peripheral organs such as the lymph node in diseases such as Alzheimer's disease, multiple sclerosis, and brain cancer. A microfluidic chip can allow organs of interest to be integrated into either the brain or peripheral components as needed to model the condition or disease of interest. According to various examples, the peripheral compartment can include: the lymph node, lung, liver, gut, heart, kidney, adipose tissue, oral or nasal mucosa, vascular bed, tumor, bone marrow, etc. Additionally in various examples, the brain compartment could contain a generic brain model or brain models for specific regions of the brain (e.g., forebrain, hippocampus, brainstem, etc.) spinal cord, or retina.

According to various examples, the present disclosure can be a type of microphysiological model that provides fluidic control over an in vitro culture system. In various examples, by fluidically connecting multiple tissue culture models in tandem, the present disclosure can allow for the communication between different organs in vivo to be studied under physiological and pathological conditions. By loading the system with cells of interest, a device of the present disclosure can be used to predict species specific and even individual specific responses.

The present disclosure provides for several design advantages including a user friendly-design, modularity, and flexible design providing for separate fluid compartments to represent distinct organs such as separate compartments for brain media and for lymph node media. According to various examples, the present disclosure can be suitable for use in clinical or biomedical laboratories, e.g. labs without trained microfluidics personnel. Such user-friendly design can enable the broadening of use of these systems for mechanistic study and clinical use and can also lower the costs of commercialization and adoption. In some embodiments, the microfluidic chip can provide for a modular design. Modularity can be preferable when each organ requires a different length of time for establishment (e.g., due to differentiation requirements, etc.). Modularity can also have the benefit of allowing easy exchange of healthy, diseased, or drug-treated compartments on demand during the experiment, and easy removal of compartments for off-chip analysis at the end of the experiment. Further, according to some examples, the microfluidic chip can interface with commercially available cell culture inserts for modular insertion and removal and provide fast and reproducible assembly of a multi-organ culture system. In some examples, the present disclosure can provide accessibility for media or tissue sampling over the course of the experiment and controllable recirculating fluid flow within each media compartments with biometric fluid flow through or across tissue models. According to various embodiments, the microfluidic chips can provide multiple media loops to address the “common media” challenge of multi-organ models, where cells in various organs can be incompatible with a shared culture media. For example, this challenge is especially relevant for neurons and T cells, which each have unique media requirements. The present disclosure allows for this distinction in media by providing, in some examples, for media separation in two distinct loops that can be maintained by a barrier. In some examples, the barrier can be modeled to represent physiologically relevant barriers such as a blood-brain barrier, a meningeal lymphatic barrier, etc.

For example, the microfluidic chips can include a loop of channels that connect wells for cell culture insert-based tissue models in line with a pump for recirculation of media and secreted molecular cues. To mimic the separation in vivo, distinct media compartments for the brain and lymph node can be provided for with passive communication across meninges barrier model. In some embodiments, the pump can be a user-friendly design that does not require external tubing, such as an on-board impeller pump.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

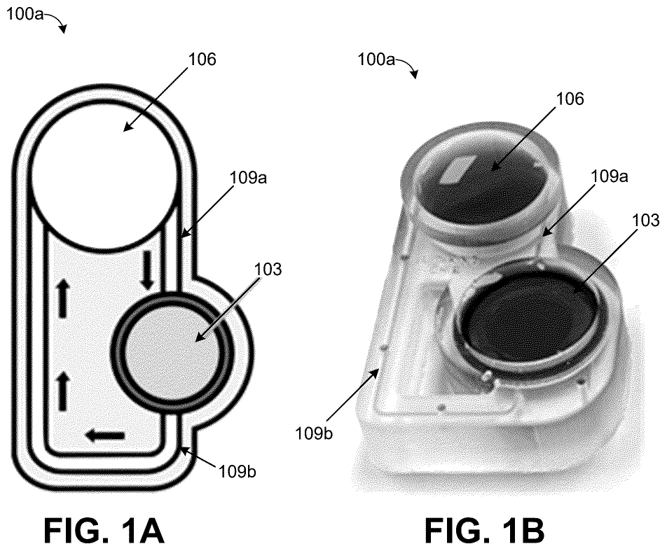

With reference to FIGS. 1A and 1B, shown in 1A is a schematic drawing and 1B is an illustration detailing an example of a single-media microfluidic chip 100a that can be used to model biological cultures in isolation according to various embodiments of the present disclosure. For example, the single media can be a brain culture of lymph node paracortex culture in isolation. While the size of the microfluidic chip 100 can change depending on the scope and size of the system being modeled, the single-media microfluidic chip 100a of FIG. 1B is approximately 26 mm by 44 mm. The single-media microfluidic chip 100a can contain a single media that can be used to confirm cell viability and function under fluid flow. While FIG. 1A shows a fluid flowing through the single-media microfluidic chip 100a in a clockwise direction, the fluid can also flow in a counterclockwise direction. According to various examples, the single-media microfluidic chip 100a can include a well 103, a pump 106, a first channel 109 (109a and 109b), and potentially other components.

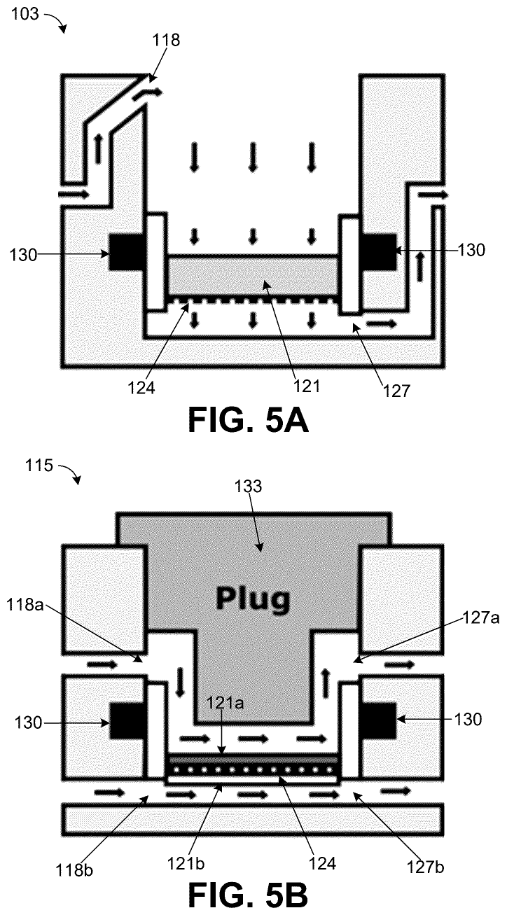

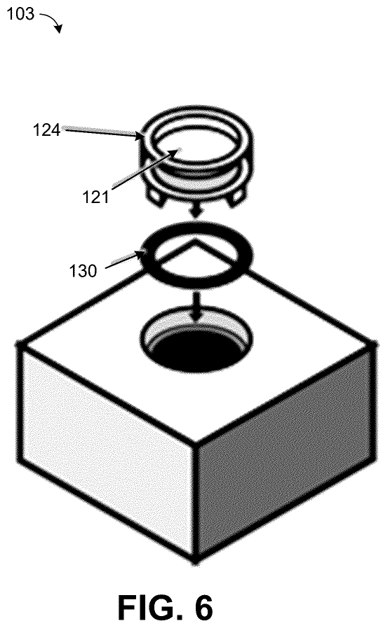

The well 103 can provide cells or tissue to be cultured. The components of a well 103 are further described in FIGS. 5A and 6. For example, the well 103 can contain a brain model, a lymph node mode, an adipose tissue model, etc. For example, a lymph node well 103 might contain an explanted lymph node tissue slice from a human or animal model or a 3D culture in a hydrogel. Similarly, the brain model well 103 might contain an explanted brain slice or a 3D culture. In the example of 3D cultures, the hydrogel can be comprised of any 3D culture matric, such as collagen, gelatin, hyaluronan, alginate, agarose, PEG (poly-ethylene glycol) and other synthetic polymers such as PLGA. These could be, for example, photocrosslinkable gels, thermosetting gels, or chemically or biochemically crosslinked or self-assembled gels. For a lymphoid culture, cells can include T cells, B cells, monocytes or macrophages, dendric cells, stroma cells, lymphatic endothelium, etc. And, for a brain culture, cells might include neurons of varied types, astrocytes, microglia, pericytes, etc. FIGS. 1A and 1B depict one well 103 but as can be appreciated through additional embodiments described herein, the microfluidic chip 100 can contain multiple wells 103. According to various examples, the size of a well 103 can be manipulated depending on the requirements of the modeled system. In the example of FIG. 1B, the well 103 has a diameter of approximately 14 mm and a height of approximately 5 mm. The well 103 can be open at the top to allow for accessibility to the media or tissue sampling over the scope of the experiment.

The pump 106 can provide for circulation throughout the single-media microfluidic chip 100a. For example, the pump 106 can provide fluidic control. In some examples, the pump 106 can include a rotating stir bar that can generate recirculating fluid flow through the connected loop of the first channel 109. In the example of FIG. 1B, the pump 106 has a diameter of approximately 15 mm and a height of approximately 9 mm. Within a pump box, one or more motor circuits can each have voltage control and readout thorough a potentiometer and voltmeter. In addition to considerations related to placement of the single-media microfluidic chip 100a (or generally microfluidic chip 100) on the pump box, a consideration of pump 106 placement in the loop is the impact of dilution of the media as it passes through the pump 106. For example, when using an impeller pump, it currently has a comparatively large volume (hundreds of microliters) compared to the volume of the media loop. Therefore, the placement of the pump 106 can be arranged to avoid placing the pump 106 between two organs that need to be in close communication, to avoid diluting the secreted signals on the first round of media circulation. This approach can also avoid disrupting transfer of circulating cells from the upstream to downstream compartment.

The first channel 109 can form a loop connecting the well 103 to the pump 106. In the example of FIGS. 1A and 1B, the first channel 109 can be composed of two portions with a first pump-well channel 109a directly connecting the pump 106 to the well 103 and a first well-pump channel 109b directly connecting the well 103 to the pump 106. For example, a fluid can flow through the first pump-well channel 109a to the well 103 and then through the first well-pump channel 109b to the pump 106, all part of the first channel 109 that comprises a single media loop. In the example of FIG. 1B, the first channel 109 can have a diameter of approximately 0.5 mm and a length of approximately 40 mm.

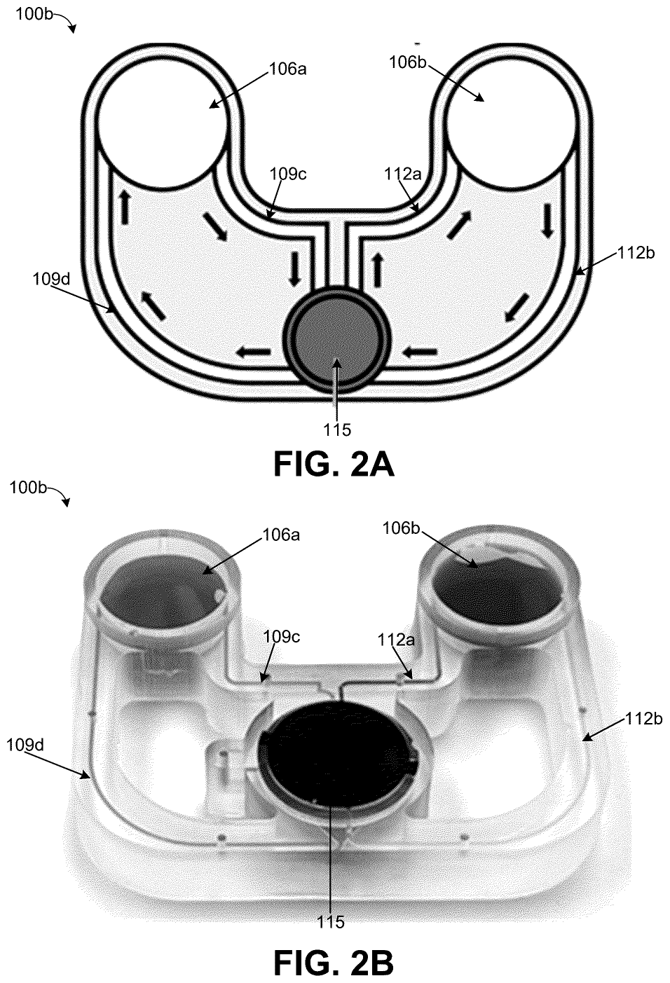

With reference to FIGS. 2A and 2B, shown in FIG. 2A is a schematic drawing and FIG. 2B is an illustration detailing an example of a barrier-only microfluidic chip 100b where the barrier can be used to model the interaction of multiple medias across biological barriers according to various embodiments of the present disclosure. For example, the barrier-only microfluidic chip 100b can model the meningeal lymphatics barrier culture under recirculating fluid flow of two distinct media loops. While FIG. 2A shows a first fluid flowing through the first media compartment in a clockwise direction and a second fluid flowing through the second media compartment in a counterclockwise direction, the directions of each fluid can be flips so that both the first fluid and the second fluid can flow in both a clockwise direction and a counterclockwise direction. According to various embodiments, the barrier-only microfluidic chip 100b can include a first pump 106a, a second pump 106b, a first channel 109 (109c and 109d), a second channel 112 (112a and 112b), a barrier well 115, and potentially other components.

The first pump 106a and the second pump 106b can provide for circulation throughout the barrier-only microfluidic chip 100b. For example, the first pump 106a can provide fluidic control of the first fluid in the first loop through the first channel 109 and the second pump 106b can provide fluidic control of the second fluid in the second loop through the second channel 112. In the example of FIG. 2B, the first pump 106a and the second pump 106b have a diameter of approximately 15 mm and a height of approximately 9 mm. In some examples, the first pump 106a and the second pump 106b can include a rotating stir bar that can generate recirculating fluid flow through the connected loop of the first channel 109 and the connected loop of the second channel 112, respectively. Within a pump box, two motor circuits can each have voltage control and readout thorough a potentiometer and voltmeter. By maintaining separate voltage control, each fluidic loop on the barrier-only microfluidic chip 100b can be recirculating at different speeds and directions depending on the needs of the modeled tissue. The placement of the first pump 106a and the second pump 106b is due to consideration of the modeled system and dilution of the medias.

The first channel 109 can form a first media loop and the second channel 112 can form a second media loop. For example, the first channel 109 can form a first loop connecting the first pump 106a to the barrier well 115 and the second channel 112 can form a second loop connecting the second pump 106b to the barrier well 115. In the example of FIGS. 2A and 2B, the first channel 109 can be composed of two portions with a first pump-barrier channel 109c directly connecting the first pump 106a to the barrier well 115 and a first barrier-pump channel 109d directly connecting the barrier well 115 to the first pump 106a. Further, the second channel 112 can be composed of two portions with a second pump-barrier channel 112b directly connecting the second pump 106b to the barrier well 115 and a second barrier-pump channel 112a directly connecting the barrier well 115 to the second pump 106b. For example, a first fluid can flow through the first pump-barrier channel 109c to the barrier well 115 and then through the first barrier-pump channel 109d to the first pump 106a, all part of the first channel 109 that comprises a first media loop. A second fluid can flow through the second pump-barrier channel 112b to the barrier well 115 and then through the second barrier-pump channel 112a the second pump 106b, all part of the second channel 112 that comprises a second media loop. In the example of FIG. 2B, the first channel 109 and the second channel 112 can have a diameter of approximately 0.5 mm and a length of approximately 60 mm.

The barrier well 115 can provide a dual media interaction. For example, the barrier well can interface with the first channel 109 and the second channel 112. In some examples, the first channel 109 can flow through a top of the barrier well 115 while the second channel 112 can flow through a bottom of the barrier well 115 and vice versa. Thus, the barrier well 115 can have two inlets and two outlets at varying positions. In some examples, the flow of the first fluid can be opposite the flow of the second fluid or the flows of both fluids can be in the same direction. According to various examples, the barrier well 115 can have two layers of cells with one layer being disposed on the bottom channel of the barrier well 115 and the other layer being disposed at the top channel of the barrier well 115. In the example of FIG. 2B, the barrier well 115 has a diameter of approximately 14 mm and a height of approximately 5 mm.

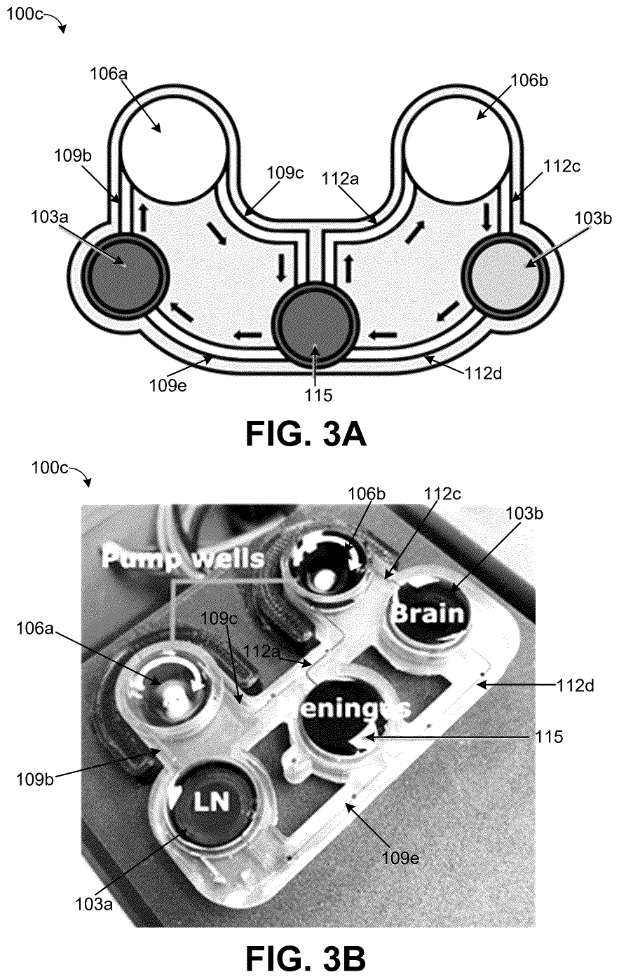

With reference to FIGS. 3A and 3B, shown in FIG. 3A is a schematic drawing and FIG. 3B is an illustration detaining an example of a multi-media microfluidic chip 100c that can be used to model biological interactions of multiple media across barriers. For example, the multi-media microfluidic chip 100c can provide for co-culture of the lymph node paracortex, meningeal lymphatics barrier, and brain tissue engineered models. The multi-media microfluidic chip 100c can include two media loops each with separate pumps that intersect at the barrier well to model biological interactions. While FIG. 3A shows a first fluid flowing through the first media compartment in a clockwise direction and a second fluid flowing through the second media compartment in a counterclockwise direction, the directions of each fluid can be flipped so that both the first fluid and the second fluid can flow in both a clockwise direction and a counterclockwise direction. According to various embodiments, the multi-media microfluidic chip 100c can include a first well 103a, a second well 103b, a first pump 106a a, a second pump 106b, a first channel 109 (109b, 109c, and 109e), a second channel 112 (112a, 112c, and 112d), a barrier well 115, and potentially other components.

The first well 103a and the second well 103b can provide cells or tissue to be cultured. For example, the first well 103a can provide a sample for the first media loop and the second well 103b can provide a sample for the second media loop. The first well 103a and the second well 103b can be open at the top to allow for accessibility to the media or tissue sampling over the scope of the experiment. According to various examples, the first well 103a can represent a lymph node model, and the second well 103b can represent a brain model.

The first pump 106a and the second pump 106b can provide for circulation throughout the multi-media microfluidic chip 100c. For example, the first pump 106a can provide fluidic control of the first fluid in the first loop through the first channel 109 and the second pump 106b can provide fluidic control of the second fluid in the second loop through the second channel 112. In some examples, the first pump 106a and the second pump 106b can include a rotating stir bar that can generate recirculating fluid flow through the connected loop of the first channel 109 and the connected loop of the second channel 112, respectively. Within a pump box, two motor circuits can each have voltage control and readout thorough a potentiometer and voltmeter. By maintaining separate voltage control, each fluidic loop on the multi-media microfluidic chip 100c can be recirculating at different speeds and directions depending on the needs of the modeled tissue. The placement of the first pump 106a and the second pump 106b is due to consideration of the modeled system and dilution of the medias. For example, the multi-media microfluidic chip 100c can be used to allow signals to drain from a brain model well 103b to the meningeal barrier well 115 and from the meningeal barrier well 115 to the lymph node well 103a, without dilution of either the first pump 106a or the second pump 106b on the first pass.

The first channel 109 can form a first media loop and the second channel 112 can form a second media loop. For example, the first channel 109 can form a first loop connecting the first well 103a to the first pump 106a to the barrier well 115 and the second channel 112 can form a second loop connecting the second pump 106b to the second well 103b to the barrier well 115. In the example of FIGS. 3A and 3B, the first channel 109 can be composed of three portions with the first well-pump channel 109b directly connecting the first well 103a to the first pump 106a, the first pump-barrier channel 109c directly connecting the first pump 106a to the barrier well 115, and a first barrier-well channel 109e directly connecting the barrier well 115 to the first well 103a. Further, the second channel 112 can be composed of three portions with a second barrier-pump channel 112a directly connecting the barrier well 115 to the second pump 106b, a second pump-well channel 112c directly connecting the second pump 106b to the second well 103b, and a second well-barrier channel 112d directly connecting the second well 103b to the barrier well 115. For example, a first fluid can flow through the first well-pump channel 109b to the first pump 106a, through the first pump-barrier channel 109c to the barrier well 115 and then through the first barrier-well channel 109e to the first well 103a, all part of the first channel 109 that comprises a first media loop. A second fluid can flow through the second barrier-pump channel 112a to the second pump 106b, through the second pump-well channel 112c to the second well 103b, and then through the second well-barrier channel 112d to the barrier well 115, all part of the second channel 112 that comprises a second media loop.

The barrier well 115 can provide a dual media interaction. For example, the barrier well can interface with the first channel 109 and the second channel 112. In some examples, the first channel 109 can flow through a top of the barrier well 115 while the second channel 112 can flow through a bottom of the barrier well 115 and vice versa. Thus, the barrier well 115 can have two inlets and two outlets at varying positions. In some examples, the flow of the first fluid can be opposite the flow of the second fluid or the flows of both fluids can be in the same direction. According to various examples, the barrier well 115 can have two layers of cells with one layer being disposed on the bottom channel of the barrier well 115 and the other layer being disposed at the top channel fo the barrier well 115. In the example of FIGS. 3A and 3B, the barrier well 115 can represent the meninges with a top layer of meningeal cells interfacing with the brain media loop and a bottom layer of lymphatic endothelial cells interfacing with the lymph node media loop.

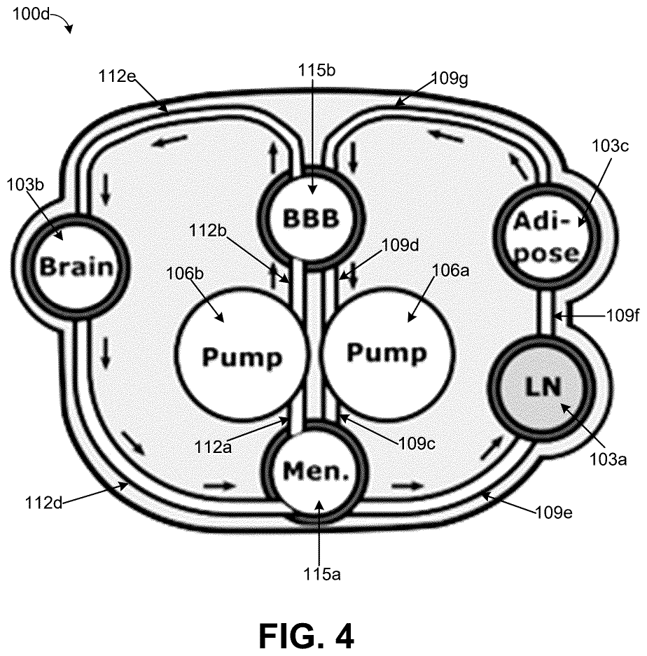

With reference to FIG. 4, shown is a schematic drawing detailing an example of a multi-media, multi-barrier microfluidic chip 100d. According to various examples, the microfluidic chip ca be expanded to culture other or additional organs across multiple barriers. FIG. 4 offers an example of an extension of wells and barriers. While FIG. 4 depicts one example with placement of additional barriers and wells designed to mimic anatomical placement, it should be understood that the microfluidic chip can be configured to place wells and barriers at various points within the loops to mimic other biological systems and fluid flows. Additionally, the fluid in either loop can flow in both a clockwise and counterclockwise direction. As shown in FIG. 4, additional modules can be added to the lymph node loop to allow the microfluidic chip to model any combination of a number of organs from outside of the central nervous system, such as lymph node, lung, heart, adipose tissue, liver, kidney, etc., thus creating a model of the brain-periphery with a model blood-brain barrier or meningeal barrier. Additionally, multiple modules in the brain loop can be included (e.g., brain and spinal cord) or modules for specific regions of the brain such as the hippocampus or the cortex, etc. FIG. 4 provides a design for a system to co-culture the brain module with both a lymph node and adipose module with the two loops separated by both a blood-brain barrier and a meningeal barrier. According to various embodiments, a multi-media, multi-barrier microfluidic chip 100d can include a first well 103a, a second well 103b, a third well 103c, a first pump 106a, a second pump 106b, a first channel 109 (109c, 109d, 109e, 109f, and 109g), a second channel 112 (112a, 112b, 112d, and 112e), a first barrier well 115a, a second barrier well 115b, and potentially other components.

The first well 103a, the second well 103b, and the third well 103c can provide cells or tissue to be cultured. For example, the first well 103a can provide a sample for the first media loop, the second well 103b can provide a sample for the second media loop and as shown in FIG. 4 the third well 103c can provide a second sample in the first media loop. The third well 103c can also be in the second media loop depending on the anatomical requirements of the modeled system. The first well 103a, the second well 103b, and the third well 103c can be open at the top to allow for accessibility to the media or tissue sampling over the scope of the experiment. According to various examples described in reference to FIG. 4, the first well 103a can represent a lymph node model, the second well 103b can represent a brain model, and the third well 103c can represent an adipose model.

The first pump 106a and the second pump 106b can provide for circulation throughout the multi-media, multi-barrier microfluidic chip 100d. For example, the first pump 106a can provide fluidic control of the first fluid in the first loop through the first channel 109 and the second pump 106b can provide fluidic control of the second fluid in the second loop through the second channel 112. In some examples, the first pump 106a and the second pump 106b can include a rotating stir bar that can generate recirculating fluid flow through the connected loop of the first channel 109 and the connected loop of the second channel 112, respectively. Within a pump box, two motor circuits can each have voltage control and readout thorough a potentiometer and voltmeter. By maintaining separate voltage control, each fluidic loop on the multi-media, multi-barrier microfluidic chip 100d can be recirculating at different speeds and directions depending on the needs of the modeled tissue. The placement of the first pump 106a and the second pump 106b is due to consideration of the modeled system and dilution of the medias. For example, the multi-media, multi-barrier microfluidic chip 100d can maintain the directionality used by the multi-media microfluidic chip 100c that allow signals to drain from a brain model well 103b to the meningeal barrier well 115 and from the meningeal barrier well 115 to the lymph node well 103a, without dilution of either the first pump 106a or the second pump 106b on the first pass. Additionally, in the multi-media, multi-barrier microfluidic chip 100d can provide a direct communication from the lymph node well 103a to the adipose tissue well 103c, and adipose tissue well 103c to the blood brain barrier well 115b, without dilution from the first pump 106a or the second pump 106b. In FIG. 4, the pump connections were inserted between the first barrier well 115a and the second barrier well 115b, since the blood brain barrier and the meningeal barrier are not in direct communication in vivo.

The first channel 109 can form a first media loop and the second channel 112 can form a second media loop. For example, the first channel 109 can form a first loop connecting the first well 103a to the third well 103c to the second barrier well 115b to the first pump 106a to the first barrier well 115a and the second channel 112 can form a second loop connecting the second well 103b to the first barrier well 115a to the second pump 106b to the second barrier well 115b. In the example of FIG. 4, the first channel 109 can be composed of five portions with the first well-well channel 109f directly connecting the first well 103a to the third well 103c, the first well-barrier channel 109g directly connecting the third well 103c to the second barrier well 115b, the first barrier-pump channel 109d directly connecting the second barrier well 115b to the first pump 106a, the first pump-barrier channel 109c directly connecting the first pump 103a to the first barrier well 115a, and the first barrier-well channel 109e directly connecting the first barrier well 115a to the first well 103a. Further, the second channel 112 can be composed of four portions with a second well-barrier channel 112d directly connecting the second well 103b to the first barrier well 115a, the second barrier-pump channel 112a directly connecting the first barrier well 115a to the second pump 106b, the second pump-barrier channel 112b directly connecting the second pump 106b to the second barrier well 115b, and the second barrier-well channel 112e directly connecting the second barrier well 115b to the second well 103b. For example, a first fluid can flow through the first well-well channel 109f to the third well 103c, through the first well-barrier channel 109g to the second barrier well 115b, through the first barrier-pump channel 109d to the first pump 106a, through the first pump-barrier channel 109c to the first barrier well 115a, and then through the first barrier-well channel 109e to the first well 103a, all part of the first channel 109 that comprises a first media loop. A second fluid can flow through the second well-barrier channel 112d to the first barrier well 115a, through the second barrier-pump channel 112a to the second pump 106b, through the second pump-barrier channel 112b to the second barrier well 115b, and then through the second barrier-well channel 112e to the second well 103b, all part of the second channel 112 that comprises a second media loop.

The first barrier well 115a and the second barrier well 115b can provide for dual media interactions. For example, both the first barrier well 115a and the second barrier well 115b can interface with the first channel 109 and the second channel 112. In some examples, the first channel 109 can flow through a top of the barrier well 115 (either the first barrier well 115a or the second barrier well 115b) while the second channel 112 can flow through a bottom of the barrier well 115 (either the first barrier well 115a or the second barrier well 115b) and vice versa. Thus, both the first barrier well 115a and the second barrier well 115b can have two inlets and two outlets at varying positions. In some examples, the flow of the first fluid can be opposite the flow of the second fluid or the flows of both fluids can be in the same direction. According to various examples, the first barrier well 115a and the second barrier well 115b can have two layers of cells with one layer being disposed on the bottom channel of the barrier well 115 (either the first barrier well 115a or the second barrier well 115b) and the other layer being disposed at the top channel of the barrier well 115 (either the first barrier well 115a or the second barrier well 115b). In the example of FIG. 4, the first barrier well 115a can represent the meninges and the second barrier well 115b can represent the blood-brain barrier, both with layers of meningeal cells interfacing with the brain media loop and layers of lymphatic endothelial cells interfacing with the lymph node media loop.

Next, in FIG. 5A shown is a cross-sectional view of one example of a well 103 according to various embodiments of the present disclosure. The arrows in FIG. 5A depict fluid flow through a well 103. For example, a fluid can flow through a channel and into the well 103 via a well inlet 118. The well inlet 118 can be positioned at the top of a well 103 to encourage fluid flow through the well 115 in a desired direction. As the fluid flows through the well 103, the fluid can flow through a layer of cells 121 and the cell culture insert 124. The layer of cells 121 can be disposed on top of the cell culture insert 124. Next, the fluid can flow from the well 103 via a well outlet 127. For example, for a brain model well 103 or a lymph node model well 103, fluid can enter the well 103 through a well inlet 118 located above a cell-laden hydrogen gel (layer of cells 121) and flow through the gel perpendicular to the cell culture insert 124 membrane and exit the well 103 through the well outlet 127 located below the cell culture insert 124. According to various embodiments of the present disclosure, the well 103 can be open at the top to allow for accessibility to the media or tissue sampling over the scope of the experiment. In some embodiments, the well 103 can further include a gasket 130. The gasket 130 can be applied to secure the cell culture insert 124 in place within the well 103. In some examples, the gasket 130 can be an O-ring.

With reference to FIG. 5B shown is a cross-sectional view of one example of a barrier well 115 according to various embodiments of the present disclosure. The arrows in FIG. 5B depict dual fluid flow through a barrier well 115. For example, a first fluid can flow through a first channel 109 and into the barrier well 115 via a first well inlet 118a. The first well inlet 118a can be positioned at the top of the barrier well 115. As the first fluid flows through the barrier well 115, the fluid can flow parallel to a first layer of cells 121a and the cell culture insert 124. The first layer of cells 121a can be disposed on top of the cell culture insert 124. Next, the first fluid can flow from the barrier well 115 via a first well outlet 127a. Similarly, the second fluid can flow through a second channel 112 and into the barrier well 115 via a second well inlet 118b. The second well inlet 118b can be positioned at the bottom of the barrier well 115. As the second fluid flows through the barrier well 115, the fluid can flow parallel to a second layer of cells 121b and the cell culture insert 124. The second layer of cells 121b can be disposed on the bottom of the cell culture insert 124. Then, the second fluid can flow through the barrier well 115 via a second well outlet 127b. While the first channel 109 was described as supplying the first well inlet 118a and the second channel 112 was described as supplying the second well inlet 118b, these can be flipped so that the second channel 112 flows into the top of the barrier well 115 and the first channel 109 flows into the bottom of the barrier well 115.

According to various examples, the meningeal lymphatics tissue engineered model can act as a barrier between the brain and lymph node media loops. In this example, the brain media can flow across the meningeal cell monolayer (a first layer of cells 121a) on the top of the cell culture insert 124 and lymph node media can flow across a lymphatic endothelial monolayer (a second layer of cells 121b) on the bottom of the cell culture insert 124. To generate this flow regime on-chip, a channel perfuses the barrier well 115 below the cell culture insert 124, while a plug 133 is used in the barrier well 115 above the cell culture insert 124 to drive fluid flow closer to the cell culture insert 124. Thus, the barrier well 115 is not always open on the top like a well 103. The barrier well 115 can also include a gasket 130 to secure the cell culture insert 124 in place within the barrier well 115. In some examples, the gasket 130 can be an O-ring. Further, in some examples, instead of a gasket, a snap-fit design can be used.

Next, in FIG. 6, shown is an exploded view of a well 103. As previously described, the well 103 can include a layer of cells 121 on top of a cell culture insert 124 that is secured in place within the well 103 via a gasket 130. Within the microfluidic chip 100, the well 103 can allow cell culture insert 124 based tissue models to be cultured without fluid leaking around the cell culture insert 124. To develop a leak-free reversible seal, the well 103 includes a gasket 130 (e.g., O-ring) to prevent leakage between a microfluidic chip 100 and the cell culture insert 124. The well 103 can also be open at the top. The open well 103 and reversible cell culture insert 124 seal enable media and tissue sampling throughout an experiment via pipetting directly out of the well 103 or removing the cell culture insert 124 entirely. In some examples, to enable fluid flow both above and below the cell culture insert 124 models, commercially available cell culture inserts 124 can be cut from a height of about 10 mm to about 3 mm and used as cell culture inserts 124.

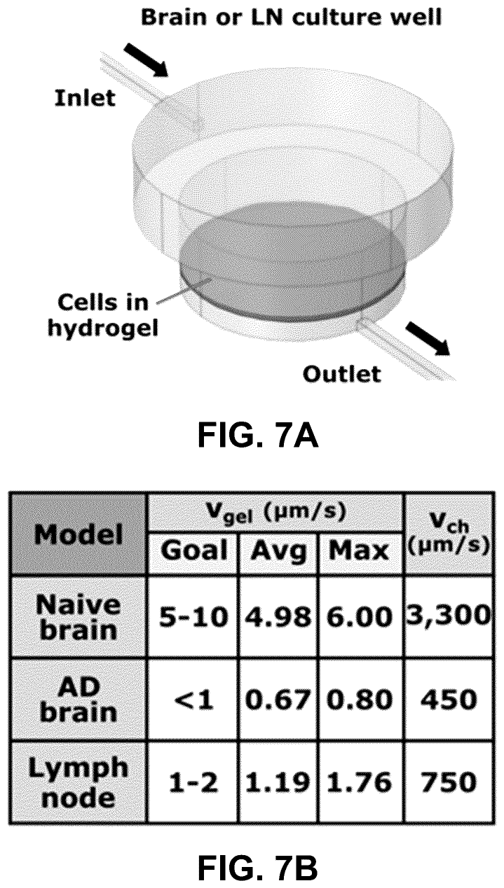

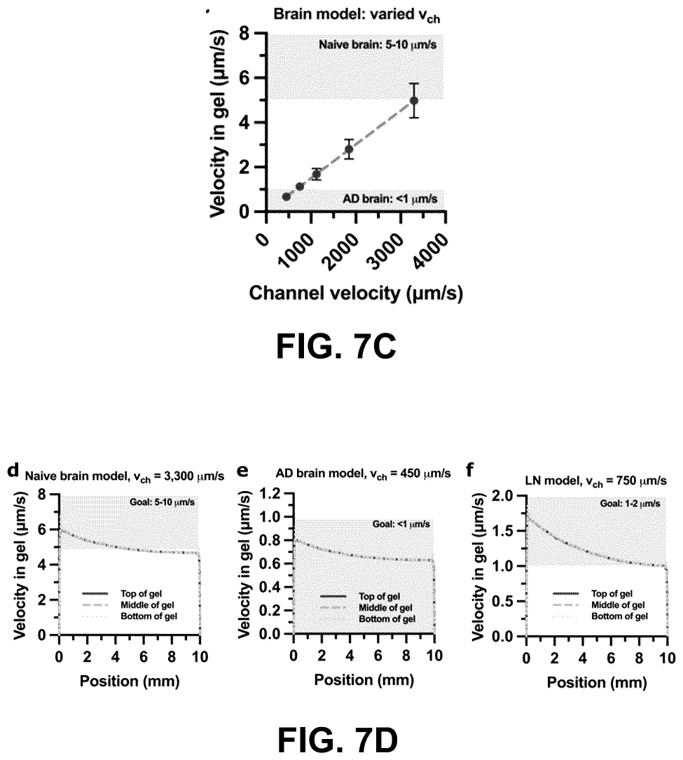

With reference to FIGS. 7A-D, shown are models and predictions of fluid flow through a well 103 according to various embodiments of the present disclosure. The speed at which fluid is moving either through a tissue (e.g., interstitial fluid flow) or through a vessel (e.g., lymphatic vasculature) can be indicative of inflammation. For example, with Alzheimer's disease, the interstitial fluid flow within brain parenchyma drastically slows sown dropping from about 5-10 μm/s to less than 1 μm/s in humans. Within the lymph node, there is limited research on the magnitude of the interstitial fluid velocity, but it is thought to be on the order of 1-2 μm/s.

When integrating tissue models into the microfluidic chip 100, a major consideration was to recapitulate the fluidic environment within or surrounding these tissues in both naïve and disease conditions. Advantages of the fluid flow within the microfluidic chip 100 is an achievable velocity through lymph node model wells 103 of about 1 to 2 μm/s, achievable velocity through brain model wells 103 of both about 5 to about 10 μm/s and less than 1 μm/s without changing the geometry of the well 103, and achievable approximate speed across the meningeal lymphatics model at all fluid speeds required for the brain and lymph node models. This desired velocity modeling is described in reference to a brain-lymph node model chip and can be adjusted based on the desired velocities and speed of the system being modeled on the microfluidic chip 100. A model of a well 103 is provided in FIG. 7A and a table of the goal, predicted average, and predicted maximum velocity through the gel (or layer of cells 121) and the corresponding channel velocities for each tissue model. The dimensions of the channels (first channel 109 and second channel 112) can be varied based on the desired velocity for the system. In the example of a brain model well 103, a range of inlet speeds can be tested to determine what channel speeds were within goal for naïve brain models and Alzheimer's disease brain models. A graph of predicted test of ranges is provided in FIG. 7C. The test found that a channel speed of 3,300 μm/s resulted in an average (4.98 μm/s) and maximum (6.00 μm/s) generally within the goal speed of about 5 to about 10 μm/s for the naïve brain model. At a much lower channel speed of 450 μm/s, the velocity in gel dropped to an average (0.67 μm/s) and maximum (0.80 μm/s) velocity both less than 1 μm/s for the Alzheimer's disease brain model. And a channel speed of 750 μm/s achieved a predicted average of (1.19 μm/s) and maximum (1.76 μm/s) velocity within the about 1 to about 2 μm/s range for the lymph node model. The channel speed compared to velocity of each desired model is provided in FIG. 7D. For all three conditions, there was little to no variability in velocity through the depth of the gel.

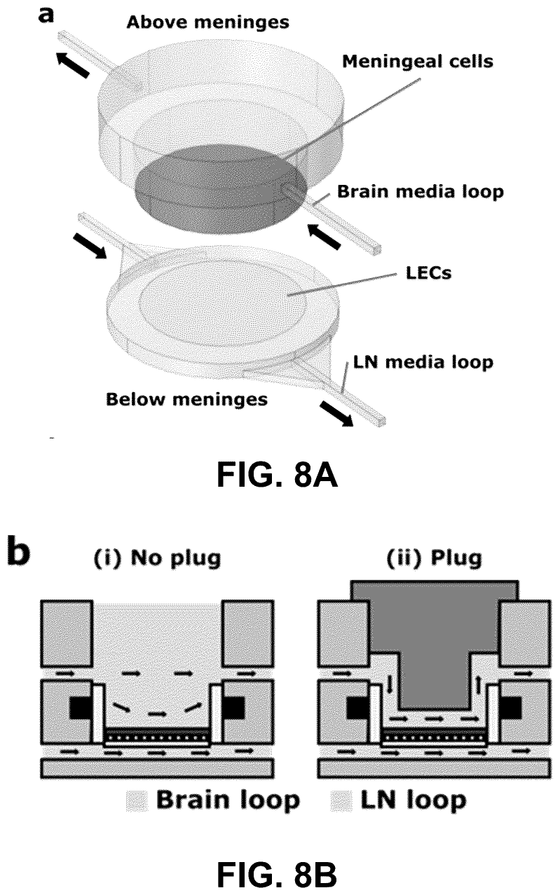

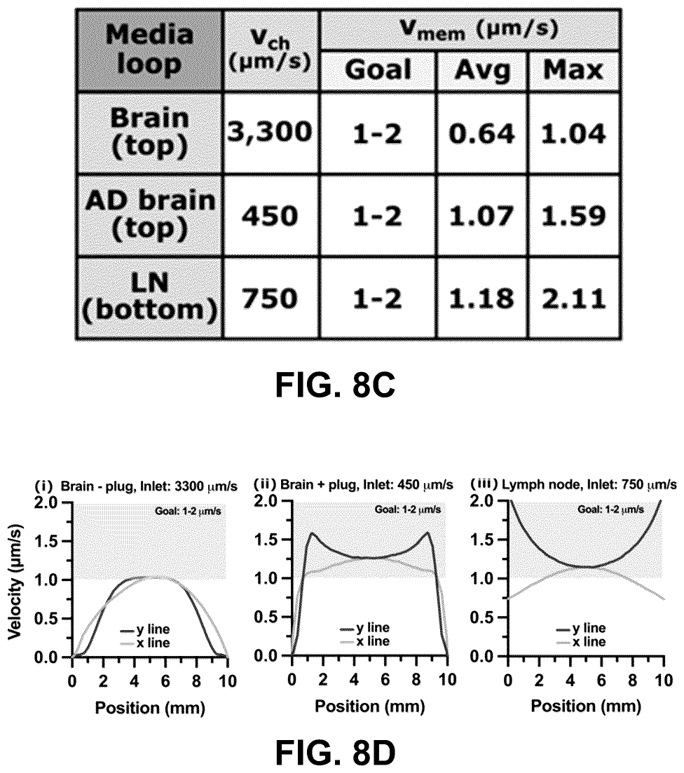

With reference to FIGS. 8A-D, shown are models and predictions of fluid flow through a barrier well 115 according to various embodiments of the present disclosure. The channel speeds were used to optimize the geometry of the meningeal lymphatic barrier well 115. In a microfluidic chip 100 with two loops and a barrier well 115, both media loops can perfuse through their respective loops and any wells 103 within the loop as well as across any barrier well 115. In this example, the channel speeds can match for the hydrogel-based tissue models and their corresponding barrier side (e.g., the brain loop can perfuse through the brain tissue engineered model and across the meningeal cell monolayer on the barrier model). For the top of the barrier well (e.g., the brain loop) and the bottom of the barrier well 115 (e.g., the lymph node loop), a goal velocity across the cell culture insert 124 was about 1 to about 2 μm/s. A model of a barrier well 115 is depicted in FIG. 8A with the model geometry split into the chamber of the barrier well 115 above the cell culture insert 124 and the chamber of the barrier well 115 below the cell culture insert 124. The chamber above the cell culture insert 124 can have an inlet and outlet on the same z plane with the layer of cells 121 in the middle of the barrier well 115. The chamber below the cell culture insert 124 can be relatively flat, where the inlet and outlet widen before reaching the cell culture insert 124 and the layer of cells 121 on the bottom of the cell culture insert 124. According to various embodiments, a plug 133 can be used to cap the top of the barrier well 115. When a plug 133 is added to the barrier well 115, the total volume is decreased and the fluid path at the top of the barrier well is driven closer to the membrane, resulting in a greater velocity across the membrane. The difference in the fluid flow without the plug 133 and with the plug 133 is shown in FIG. 8B. As discussed above, there is a significant difference in channel speeds (FIG. 8C) required between the naïve brain model (3,300 μm/s) and the Alzheimer's disease brain model (450 μm/s). The goal velocities can be achieved from these various models by using a plug 133 free barrier well for the naïve brain model and adding the plug 133 for the Alzheimer's disease brain model. For the chamber below the cell culture insert 124, the volume was optimized to allow for the goal velocity of about 1 to about 2 μm/s with a channel speed of 750 μm/s for the lymph node model. A graph of the velocity of the various models is shown in FIG. 8D.

There are many barriers in vivo that separate different tissues fluidically (e.g., the blood-brain barrier, the meninges for the brain, the gut mucosal barrier, etc.). While these barriers aim to separate different regions of the body, cross-talk across the barrier can be required for each part of the body to work in concert. This is achieved on the microfluidic chip 100 by separating media compartments with one or more semi-permeable barrier well 115. At least one aim of the barrier well 115 is to allow molecules or cells to cross, depending on the modeled barrier and inflammatory state, while keeping media types distinct enough to maintain cell viability.

### Aspects

Aspect 1. A microfluidic device, comprising: a first pump; a first well comprising a first cell culture insert disposed in the first well; and a first channel connecting the first pump to the first well.

Aspect 2. The microfluidic device of Aspect 1, further comprising: a second pump; a second well, comprising a second cell culture insert disposed in the second well; a second channel connecting the second pump to the second well; and a barrier well disposed along both the first channel between the first pump and the first well and the second channel between the second well and the second pump.

Aspect 3. The microfluidic device of Aspect 2, wherein the barrier well comprises: a barrier cell culture insert disposed in the barrier well; a first barrier inlet connecting the barrier well to the first channel; a second barrier inlet connecting the barrier well to the second channel; a first barrier outlet connecting the barrier well to the first channel; and a second barrier outlet connecting the barrier well to the second channel.

Aspect 4. The microfluidic device of Aspect 3, wherein the first well further comprises a first gasket to secure the first cell culture in place within the first well, the second well further comprises a second gasket to secure the second cell culture insert in place within the second well, and the barrier well further comprises a barrier gasket to secure the barrier cell culture insert in place within the barrier well.

Aspect 5. The microfluidic device of Aspect 4, wherein at least one of the first gasket, the second gasket, or the barrier gasket is an O-ring.

Aspect 6. The microfluidic device of Aspect 3, further comprising: a third well disposed along the first channel between the first well and the first pump, wherein the third well, comprises: a third cell culture insert disposed in the third well; and a third gasket to secure the third cell culture in place within the third well; and a second barrier well disposed along both the first channel between the third well and the first pump and the second channel between the second pump and the second well.

Aspect 7. A method, comprising flowing a first fluid through a microfluidic chip, the microfluidic chip, comprising: a first pump configured to pump the first fluid through the microfluidic chip; a first well, the first well comprising a first cell culture insert disposed in the first well; and a first channel connecting the first pump and the first well, wherein the first pump is in fluidic communication with the first well via the first fluid.

Aspect 8. The method of Aspect 7, further comprising flowing a second fluid through the microfluidic chip wherein the microfluidic chip further comprises: a second pump configured to pump the second fluid through the microfluidic chip; a second well, comprising a second cell culture insert disposed in the second well; a second channel connecting the second pump to the second well, wherein the second pump is in fluidic communication with the second well via the second fluid; and a barrier well is disposed along the first channel between the first pump and the first well and along the second channel between the second well and the second pump, wherein the barrier well is in fluidic communication with the first pump and the first well via the first fluid in the first channel and in fluidic communication with the second well and the second pump via the second fluid in the second channel.

Aspect 9. The method of Aspect 8, wherein the barrier well comprises: a plug to secure a top of the barrier well; a barrier cell culture insert disposed in the barrier well; a first barrier inlet in fluidic communication with the first channel to connect the first pump to the top of the barrier well; a second barrier inlet in fluidic communication with the second channel to connect the second well to a bottom of the barrier well; a first barrier outlet in fluidic communication with the first channel to connect the top of the barrier well to the first well; and a second barrier outlet in fluidic communication with the second channel to connect the bottom of the barrier well to the second pump.

Aspect 10. The method of Aspect 9, wherein the first well further comprises a first layer of cells disposed on a top of the first cell culture insert; the second well further comprises a second layer of cells disposed on a top of the second cell culture insert; and the barrier well further comprises a third layer of cells disposed on a top of the barrier cell culture insert and a fourth layer of cells disposed on a bottom of the barrier cell culture insert.

Aspect 11. The method of Aspect 10, wherein the first layer of cells are brain cells, the second layer of cells are lymph node cells, the third layer of cells are meningeal cells, and the fourth layer of cells are lymphatic endothelial cells.

Aspect 12. The method of Aspect 9, wherein the first well further comprises a first gasket to secure the first cell culture in place within the first well, the second well further comprises a second gasket to secure the second cell culture insert in place within the second well, and the barrier well further comprises a barrier gasket to secure the barrier cell culture insert in place within the barrier well.

Aspect 13. The method of Aspect 12, wherein at least one of the first gasket, the second gasket, or the barrier gasket is an O-ring.

Aspect 14. The method of Aspect 9, wherein the microfluidic chip further comprises: a third well in fluidic communication with the first well and the first pump via the first channel, the third well comprising, comprising: a third cell culture insert disposed in the third well; and a third gasket to secure the third cell culture in place within the third well; and a second barrier well in fluidic communication with the third well and the first pump via the first channel and the second pump and the second well via the second channel.

Aspect 15. A microfluidic device, comprising: a first pump; a second pump; a barrier well, comprising a barrier cell culture insert disposed in the barrier well; a first channel connecting the first pump to a first barrier inlet and connecting a first barrier outlet to the first pump; and a second channel connecting the second pump to a second barrier inlet and connecting a second barrier outlet to the second pump.

Aspect 16. The microfluidic device of Aspect 15, wherein the barrier well further comprises a plug to secure a top of the barrier well.

Aspect 17. The microfluidic device of Aspect 15, wherein the first barrier inlet and the first barrier outlet are disposed at a top of the barrier well and the second barrier inlet and the second barrier outlet are disposed at a bottom of the barrier well.

Aspect 18. The microfluidic device of Aspect 15, wherein the first barrier inlet and the first barrier outlet are disposed at a bottom of the barrier well and the second barrier inlet and the second barrier outlet are disposed at a top of the barrier well.

Aspect 19. The microfluidic device of Aspect 15, further comprising: a first well, comprising a first cell culture insert disposed in the first well, wherein the first well is disposed along the first channel between the barrier well and the first pump; and a second well, comprising a second cell culture insert disposed in the second well, wherein the second well is disposed along the second channel between the second pump and the barrier well.

Aspect 20. The microfluidic device of Aspect 19, wherein the first well further comprises: a first well inlet that connects a top of the first well to the barrier well via the first channel; and a first well outlet that connects a bottom of the first well to the pump via the first channel.

Aspect 21. The microfluidic device of Aspect 19, wherein the second well further comprises: a second well inlet that connects a top of the second well to the pump via the second channel; and a second well outlet that connects a bottom of the second well to barrier well via the second channel.

Aspect 22. The microfluidic device of Aspect 19, wherein the barrier well further comprises a barrier gasket to secure the barrier cell culture insert in place within the barrier well, the first well further comprises a first gasket to secure the first cell culture insert in place within the first well, and the second well further comprises a second gasket to secure the second cell culture insert in place within the second well.

Aspect 23. The microfluidic device of Aspect 22, wherein at least one of the first gasket, the second gasket, or the barrier gasket is an O-ring.

Aspect 24. The microfluidic device of Aspect 19, further comprising a third well, comprising: a third cell culture insert disposed in the third well; and a third gasket to secure the third cell culture insert in place within the third well, wherein the third well is disposed along the first channel between the first well and the first pump; and
a second barrier well, comprising a plug to secure a top of the second barrier well; and a second barrier cell culture insert disposed in the second barrier well, wherein the second barrier well is disposed along both the first channel between the third well and the first pump and the second channel between the second pump and the second well.

Aspect 25. A system, comprising: a microfluidic chip, comprising: a first pump configured to pump a first fluid flowing through the microfluidic chip via a first channel; a second pump configured to pump a second fluid flowing through the microfluidic chip via a second channel; a barrier well, comprising a barrier cell culture insert disposed in the barrier well; the first channel connecting the first pump to a first barrier inlet and connecting a first barrier outlet to the first pump; and the second channel connecting the second pump to a second barrier inlet and connecting a second barrier outlet to the second pump.

Aspect 26. The system of Aspect 25, wherein the barrier well further comprises a plug to secure a top of the barrier well.

Aspect 27. The system of Aspect 25, wherein the first barrier inlet and the first barrier outlet are disposed at a top of the barrier well and the second barrier inlet and the second barrier outlet are disposed at a bottom of the barrier well.

Aspect 28. The system of Aspect 25, wherein the first barrier inlet and the first barrier outlet are disposed at a bottom of the barrier well and the second barrier inlet and the second barrier outlet are disposed at a top of the barrier well.

Aspect 29. The system of Aspect 25, wherein the microfluidic chip further comprises: a first well, comprising: a first cell culture insert disposed in the first well, wherein the first well is in fluidic communication with the barrier well and the first pump via the first fluid in the first channel; and a second well, comprising: a second cell culture insert disposed in the second well, wherein the second well is in fluidic communication with the second pump and the barrier well via the second fluid in the second channel.

Aspect 30. The system of Aspect 29, wherein the first well further comprises: a first well inlet that is in fluidic communication with a top of the first well to the barrier well via the first fluid in the first channel; and a first well outlet that is in fluidic communication with a bottom of the first well to the pump via the first fluid in the first channel.

Aspect 31. The system of Aspect 29, wherein the second well further comprises: a second well inlet that is in fluidic communication with a top of the second well to the pump via the second fluid in the second channel; and a second well outlet that is in fluidic communication with a bottom of the second well to barrier well via the second fluid in the second channel.

Aspect 32. The system of Aspect 29, wherein the first well further comprises a first layer of cell disposed on a top of the first cell culture insert; the second well further comprises a second layer of cells disposed on a top of the second cell culture insert; and the barrier well further comprises a third layer of cells disposed on a top of the barrier cell culture insert and a fourth layer of cells disposed on a bottom of the barrier cell culture insert.

Aspect 33. The system of Aspect 32, wherein the first layer of cells are brain cells, the second layer of cells are lymph node cells, the third layer of cells are meningeal cells, and the fourth layer of cells are lymphatic endothelial cells.

Aspect 34. The system of Aspect 29, wherein the barrier well further comprises a barrier gasket to secure the barrier cell culture insert in place within the barrier well, the first well further comprises a first gasket to secure the first cell culture insert in place within the first well, and the second well further comprises a second gasket to secure the second cell culture insert in place within the second well.

Aspect 35. The system of Aspect 34, wherein at least one of the first gasket, the second gasket, or the barrier gasket is an O-ring.

Aspect 36. The system of Aspect 29, wherein the microfluidic chip further comprises: a third well, comprising: a third cell culture insert disposed in the third well; and a third gasket to secure the third cell culture insert in place within the third well, wherein the third well is in fluidic communication with the first well and the first pump via the first fluid in the first channel; and a second barrier well, comprising: a plug to secure a top of the second barrier well; and a second barrier cell culture insert disposed in the second barrier well, wherein the second barrier well is in fluidic communication with the third well and the first pump via the first fluid in the first channel and with the second pump and the second well via the second fluid in the second channel.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A microfluidic device, comprising:
a first pump;
a first well comprising a first cell culture insert disposed in the first well; and
a first channel connecting the first pump to the first well.

2. The microfluidic device of claim 1, further comprising:
a second pump;
a second well, comprising a second cell culture insert disposed in the second well;
a second channel connecting the second pump to the second well; and
a barrier well disposed along both the first channel between the first pump and the first well and the second channel between the second well and the second pump.

3. The microfluidic device of claim 2, wherein the barrier well comprises:
a barrier cell culture insert disposed in the barrier well;
a first barrier inlet connecting the barrier well to the first channel;
a second barrier inlet connecting the barrier well to the second channel;
a first barrier outlet connecting the barrier well to the first channel; and
a second barrier outlet connecting the barrier well to the second channel.

4. The microfluidic device of claim 3, wherein the first well further comprises a first gasket to secure the first cell culture in place within the first well, the second well further comprises a second gasket to secure the second cell culture insert in place within the second well, and the barrier well further comprises a barrier gasket to secure the barrier cell culture insert in place within the barrier well.

5. The microfluidic device of claim 4, wherein at least one of the first gasket, the second gasket, or the barrier gasket is an O-ring.

6. The microfluidic device of claim 3, further comprising:
a third well disposed along the first channel between the first well and the first pump, wherein the third well, comprises:
a third cell culture insert disposed in the third well; and
a third gasket to secure the third cell culture in place within the third well; and
a second barrier well disposed along both the first channel between the third well and the first pump and the second channel between the second pump and the second well.

7. A method, comprising:
flowing a first fluid through a microfluidic chip, the microfluidic chip, comprising:
a first pump configured to pump the first fluid through the microfluidic chip;
a first well, the first well comprising a first cell culture insert disposed in the first well; and
a first channel connecting the first pump and the first well, wherein the first pump is in fluidic communication with the first well via the first fluid.

8. The method of claim 7, further comprising:
flowing a second fluid through the microfluidic chip, wherein the microfluidic chip further comprises:
a second pump configured to pump the second fluid through the microfluidic chip;
a second well, comprising a second cell culture insert disposed in the second well;
a second channel connecting the second pump to the second well, wherein the second pump is in fluidic communication with the second well via the second fluid; and
a barrier well is disposed along the first channel between the first pump and the first well and along the second channel between the second well and the second pump, wherein the barrier well is in fluidic communication with the first pump and the first well via the first fluid in the first channel and in fluidic communication with the second well and the second pump via the second fluid in the second channel.

9. The method of claim 8, wherein the barrier well comprises:
a plug to secure a top of the barrier well;
a barrier cell culture insert disposed in the barrier well;
a first barrier inlet in fluidic communication with the first channel to connect the first pump to the top of the barrier well;
a second barrier inlet in fluidic communication with the second channel to connect the second well to a bottom of the barrier well;
a first barrier outlet in fluidic communication with the first channel to connect the top of the barrier well to the first well; and
a second barrier outlet in fluidic communication with the second channel to connect the bottom of the barrier well to the second pump.

10. The method of claim 9, wherein the first well further comprises a first layer of cells disposed on a top of the first cell culture insert; the second well further comprises a second layer of cells disposed on a top of the second cell culture insert; and the barrier well further comprises a third layer of cells disposed on a top of the barrier cell culture insert and a fourth layer of cells disposed on a bottom of the barrier cell culture insert.

11. The method of claim 10, wherein the first layer of cells are brain cells, the second layer of cells are lymph node cells, the third layer of cells are meningeal cells, and the fourth layer of cells are lymphatic endothelial cells.

12. The method of claim 9, wherein the first well further comprises a first gasket to secure the first cell culture in place within the first well, the second well further comprises a second gasket to secure the second cell culture insert in place within the second well, and the barrier well further comprises a barrier gasket to secure the barrier cell culture insert in place within the barrier well.

13. The method of claim 9, wherein the microfluidic chip further comprises:
a third well in fluidic communication with the first well and the first pump via the first channel, the third well comprising, comprising:
a third cell culture insert disposed in the third well; and
a third gasket to secure the third cell culture in place within the third well; and
a second barrier well in fluidic communication with the third well and the first pump via the first channel and the second pump and the second well via the second channel.

14. A system, comprising:
a microfluidic chip, comprising:
a first pump configured to pump a first fluid flowing through the microfluidic chip via a first channel;
a second pump configured to pump a second fluid flowing through the microfluidic chip via a second channel;
a barrier well, comprising a barrier cell culture insert disposed in the barrier well;
the first channel connecting the first pump to a first barrier inlet and connecting a first barrier outlet to the first pump; and
the second channel connecting the second pump to a second barrier inlet and connecting a second barrier outlet to the second pump.

15. The system of claim 14, wherein the barrier well further comprises a plug to secure a top of the barrier well.

16. The system of claim 14, wherein the microfluidic chip further comprises:
a first well, comprising:
a first cell culture insert disposed in the first well, wherein the first well is in fluidic communication with the barrier well and the first pump via the first fluid in the first channel; and
a second well, comprising:
a second cell culture insert disposed in the second well, wherein the second well is in fluidic communication with the second pump and the barrier well via the second fluid in the second channel.

17. The system of claim 16, wherein the first well further comprises:
a first well inlet that is in fluidic communication with a top of the first well to the barrier well via the first fluid in the first channel; and
a first well outlet that is in fluidic communication with a bottom of the first well to the pump via the first fluid in the first channel.

18. The system of claim 16, wherein the second well further comprises:
a second well inlet that is in fluidic communication with a top of the second well to the pump via the second fluid in the second channel; and
a second well outlet that is in fluidic communication with a bottom of the second well to barrier well via the second fluid in the second channel.

19. The system of claim 16, wherein the first well further comprises a first layer of cells disposed on a top of the first cell culture insert; the second well further comprises a second layer of cells disposed on a top of the second cell culture insert; and the barrier well further comprises a third layer of cells disposed on a top of the barrier cell culture insert and a fourth layer of cells disposed on a bottom of the barrier cell culture insert.

20. The system of claim 16, wherein the microfluidic chip further comprises:
a third well, comprising:
a third cell culture insert disposed in the third well; and
a third gasket to secure the third cell culture insert in place within the third well, wherein the third well is in fluidic communication with the first well and the first pump via the first fluid in the first channel; and
a second barrier well, comprising
a plug to secure a top of the second barrier well; and
a second barrier cell culture insert disposed in the second barrier well,
wherein the second barrier well is in fluidic communication with the third well and the first pump via the first fluid in the first channel and with the second pump and the second well via the second fluid in the second channel.
